(19) United States
(12) Patent Application Publication
(10) Pub. No.: US 2023/0203446 A1
(43) Pub. Date: Jun. 29, 2023

(54) METHOD FOR WASHING AND FINISHING A GROWN CELL MASS

(71) Applicant: Upside Foods, Inc., Berkeley, CA (US)

(72) Inventors: Konrad Müller-Auffermann, Emeryville, CA (US); Stephen K. Hsu, Albany, CA (US); Kalle Lukyan Johnson, Orinda, CA (US); Matthew Leung, Richmond, VA (US); Jayanthi Lakkyreddy, San Carlos, CA (US); Morgan Laurence Rease, Emeryville, CA (US); Jessica Joslin, Emeryville, CA (US)

(21) Appl. No.: 18/146,541

(22) Filed: Dec. 27, 2022

Related U.S. Application Data
(60) Provisional application No. 63/294,703, filed on Dec. 29, 2021.
(60) Provisional application No. 63/294,700, filed on Dec. 29, 2021.

(51) Int. Cl. C12N 5/077; C12N 5/00; A23L 13/00

(52) U.S. Cl. CPC C12N 5/0652; A23L 13/00; C12N 5/0018; C12N 2500/00; C12N 2527/00

(57) ABSTRACT

This disclosure relates to methods of washing cells from a grown cell mass to remove cell culture media and enriching the cells with a finishing media. The disclosed method includes growing a cell mass in cell culture media and then collecting and washing the grown cell mass with a series of wash buffers or a gradient wash buffer that changes over time. While the cell culture media contains nutrients and components beneficial for cell growth, cells grown in cell culture media often have off flavors, off aromas, poor color, poor salt/minerality compositions, and other shortcomings. Accordingly, the disclosed method comprises removing cell culture media remnants from a grown cell mass using a single or a series of wash media. The grown cell mass is further washed with a finishing or enrichment buffer to further improve sensory aspects and nutritional composition of the grown cell mass.

### CROSS-REFERENCE TO RELATED APPLICATIONS

The present application claims the benefit of, and priority to, U.S. Provisional Application No. 63/294,703, entitled “Method for Washing and Finishing a Grown Cell Mass,” filed on Dec. 29, 2021. The present application also claims the benefit of, and priority to U.S. Provisional Application No. 63/294,700, entitled “Method for Pressurizing Cells Grown in Hydrogel to Induce Hypertrophy,” filed on Dec. 29, 2021. The aforementioned applications are hereby incorporated by reference in their entirety.

### BACKGROUND

As the world’s population continues to grow, cell-based or cultured meat products for consumption have emerged as an attractive alternative (or supplement) to conventional meat from animals. For instance, cell-based, cultivated, or cultured meat represents a technology that could address the specific dietary needs of humans. Because the cells for cell-based meat are lab grown, lab methods of preparing cell-based meat can modify the profile of essential amino acids and fats as well as enrich the meat in vitamins, minerals, and bioactive compounds. In some cases, cell-based-meat products can be prepared from a combination of cultivated adherent and suspension cells derived from a non-human animal that facilitates such modifications and enrichment.

In addition to addressing dietary needs, cell-based-meat products help alleviate several drawbacks linked to conventional meat products for humans, livestock, and the environment. For instance, conventional meat production involves controversial practices associated with animal husbandry and slaughter. Other drawbacks associated with conventional meat production include low conversion of caloric input to edible nutrients, microbial contamination of the product, emergence and propagation of veterinary and zoonotic diseases, relative natural resource requirements, and resultant industrial pollutants, such as greenhouse gas emissions and nitrogen waste streams.

Despite advances in creating cell-based-meat products, existing methods for cultivating and processing cell-based-meat products face several shortcomings, such as challenges or failures to mimic the flavors and nutritional composition of conventional meat. Existing methods often result in final cell-based-meat products having flavors, aromas, or colors that are uncharacteristic of (or different from) slaughtered meat taken from an animal. As one example, existing methods may include growing meat cells in growth media with compounds that negatively impact the flavor profile of meat cells. Growth media often contains aromatic amino acids that may degrade or oxidize into a complex mixture of primary and secondary compounds including aldehydes, alcohols, and ketones. The aldehydes, ketones, and alcohols can lead to undesirable aromas (e.g., a “wet dog” smell) or flavors, such as rancid odors or flavors. Beyond differing aromas or flavors, existing methods sometimes produce cell-based-meat products with a consistent and near homogenous pink color that lacks some of the natural reds or other shades of slaughtered meat.

Furthermore, meat cells cultivated using existing methods often lack nutritional composition relative to conventional meat. Conventional meat typically has highly digestible proteins with amino acids, vitamins, and minerals. Due, in part, to the absence of blood providing nutrients, existing methods often create cell-based-meat products deficient in many nutrients present in conventional meat. Further to the point, cell-based-meat products soaked in growth media can also exhibit excessively high osmolarity or salt-minerality compositions uncharacteristic of slaughtered meat.

These, along with additional problems and issues are present in existing methods for cultivating cell-based-meat products.

### BRIEF SUMMARY

This disclosure generally describes methods of washing cells from a grown cell mass to remove cell culture media and enriching the cells with a finishing media. In particular, the disclosed method includes growing a cell mass in cell culture media and then collecting and washing the grown cell mass with a series of wash buffers or a gradient wash buffer that changes over time. One such wash buffer may displace or remove cell culture media. Another wash buffer may take the form of a finishing buffer that improves texture and the nutritional composition of the cells of the grown cell mass. Additionally or alternatively, the disclosed method can use a gradient wash buffer by decreasing concentrations of wash media and increasing concentrations of finishing media over time. Whether applied separately or as a gradient mixture, the disclosed method can wash a grown cell mass with wash media and rinse the grown cell mass with finishing media to remove or dilute cell culture media remnants, oxidize unsuitable flavors and aromas, improve color, and/or enrich the cell grown mass with nutrients.

### BRIEF DESCRIPTION OF THE DRAWINGS

The detailed description refers to the drawings briefly described below.

FIG. 1 illustrates an overview diagram of washing and enriching a grown cell mass using washing media and enrichment media in accordance with one or more embodiments of the present disclosure.

FIG. 2 illustrates example characteristics of the completion of a cell threshold proliferation phase in accordance with one or more embodiments of the present disclosure.

FIG. 3 illustrates a multiple wash method and a gradient wash method in accordance with one or more embodiments of the present disclosure.

FIG. 4 illustrates an example system comprising various components for washing and enriching a grown cell mass in accordance with one or more embodiments of the present disclosure.

FIG. 5 illustrates example washing media compositions and example enrichment media compositions in accordance with one or more embodiments of the present disclosure.

FIG. 6 illustrates flowing a first exchange media and a second exchange media over a cell of a cell mass in accordance with one or more embodiments of the present disclosure.

FIG. 7 illustrates a series of acts for washing and enriching a grown cell mass using a washing media and an enrichment media in accordance with one or more embodiments of the present disclosure.

FIG. 8 illustrates a series of acts for washing and enriching a grown cell mass using a gradient washing media in accordance with one or more embodiments of the present disclosure.

FIG. 9 illustrates a series of acts for flushing a cell mass with exchange media in accordance with one or more embodiments of the present disclosure.

### DETAILED DESCRIPTION

This disclosure describes one or more embodiments of a method for washing and preparing a grown cell mass to improve the texture, flavor, and aroma of a cell-based-meat product. The disclosed method includes growing a cell mass and harvesting the grown cell mass after the grown cell mass completes a threshold growth phase. The grown cell mass can be collected and separated from cell culture media. Whether applied in separate wash medias or as a gradient of wash media, the disclosed method can wash the grown cell mass with a first wash buffer composition that displaces residual cell culture media and then rinse the grown cell mass with a finishing buffer that contains nutrients to enrich the grown cells.

To illustrate, the disclosed method comprises growing a cell mass in cell culture media, removing the grown cell mass from the cell culture media, washing the grown cell mass with a washing media to flush out remaining cell culture media, and rinsing the grown cell mass with an enrichment media comprising nutrients favorable for human consumption. In some implementations, rather than using a separate washing media and a separate enrichment media, the disclosed method comprises washing the grown cell mass with a gradient washing media by decreasing concentrations of washing media and increasing concentrations of enrichment media.

As mentioned, the disclosed method includes growing a cell mass. Cells may be grown within a growth environment, such as a bioreactor. More specifically, suspension cells and/or adherent cells may be grown in the presence of cell culture media. As explained further below, cell culture media comprises a source of energy and compounds for cells to grow.

After growing a cell mass, the disclosed method further comprises washing cells when they have completed threshold proliferation phase. Generally, cells complete a threshold proliferation phase when the cells have slowed or stopped exponential proliferation. Cells that have completed a threshold proliferation phase may have different characteristics. For example, the disclosed method can include evaluating cell density, cell metabolism, compounds present within cell culture media, and other factors to determine whether cells have completed a threshold proliferation or threshold growth phase and become a grown cell mass.

As indicated above, the grown cell mass may be washed using a washing media. In some embodiments, the disclosed method comprises using a series of washing media. Whether applied in separate wash medias or as a gradient of wash media, the washing media can displace or remove the cell culture media from the grown cell mass by, for example, flowing or spraying the washing media over the grown cell mass, immersing the grown cell mass in the washing media, or using some other suitable washing approach. After or as part of washing, the disclosed method can further agitate the grown cell mass and the washing media.

After or as part of washing a grown cell mass, the disclosed method may also enrich the grown cell mass using an enrichment media. Generally, the enrichment media contains vitamins, amino acids, antioxidants, fats (e.g., oils or adipocytes), and/or other compounds to improve the quality of a cell-based-meat product. In some cases, the enrichment media also adds or changes a color of the grown cell mass. The enrichment media acts as a final or finishing treatment for the grown cells in a grown cell mass to increase the nutritional value of the grown cells while also balancing components within the washed grown cell mass.

When washing with a washing media and/or enrichment media, the grown cell mass may be washed using a series of media or a gradient washing media. For example, the disclosed method can include washing cells with the washing media, removing the washing media, and adding the enrichment media. In other embodiments, the disclosed method includes washing the cells with a gradient washing media where the gradient washing media is flowed over the cells for a set time and the composition of the gradient washing media varies with time. For example, the gradient washing media may be applied by decreasing concentrations of washing media and increasing concentrations of enrichment media over time.

The disclosed method provides several benefits relative to unprocessed cell cultures or other existing and unprocessed cell-based meats. By washing the grown cell mass using a washing media, the disclosed method removes cell culture media and compounds that impart flavors or aromas to the cells that are uncharacteristic of (or different from) slaughtered meat. The washing media can also adjust the ion and pH balance within the grown cell mass to prepare the grown cell mass for the enrichment media.

In addition to improving flavors or aromas of cell-based meats, the disclosed method also improves the nutritional composition of a cell-based-meat product relative to existing and unprocessed cell-based meats. In particular, the disclosed method comprises rinsing the grown cell mass with an enrichment media comprising nutrients—after cells have been grown in cell culture media and had some or all of the cell culture media washed off. In some embodiments, the grown cell mass uptakes vitamins, amino acids, antioxidants, fats, and/or other beneficial compounds from the enrichment media. The use of the enrichment media improves the nutritional composition of the finished cell-based-meat product.

As illustrated by the foregoing discussion, the present disclosure utilizes a variety of terms to describe features and advantages of the disclosed method. Additional detail is now provided regarding the meaning of such terms. For example, as used herein, the term “cell mass” refers to a mass comprising cells of meat. In particular, a cell mass refers to cells of cultured meat gathered into a collective mass. As discussed below, a cell mass may comprise different cell types, such as one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, or other similar cell types. For example, a cell mass can include a cell sheet of cultured meat growing within an enclosure, such as a chamber, housing, container, etc.

Relatedly, the term “grown cell mass” refers to a cell mass comprising one or more grown cells. For instance, a grown cell mass includes a group of cells that have been nourished by a growth medium (e.g., a cell culture medium) to grow during a growing time period. In some cases, a grown cell mass includes a cell mass that has finished a growing time period.

As further used herein, the term “cells” refers to individual cells of meat. In particular, cells may comprise different cell types, such as one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, or other similar cell types. Furthermore, cells may comprise different types of progenitor cells, including myogenic progenitors, adipogenic progenitors, mesenchymal progenitors, or other types of progenitor cells.

As further used herein, the term “cell culture media” or “growth media” refers to a liquid or gel comprising compounds that support the growth of cells. In particular, cell culture media comprise sources of energy and compounds to regulate the cell cycle. For example, a cell culture media can contain amino acids, vitamins, inorganic salts, glucose, dissolved gases, serum, growth factors, hormones, and attachment factors. The cell culture media may also help maintain pH and osmolarity during cell growth and proliferation.

As used herein, the term “washing media” refers to a liquid for washing cells after cells have grown into a grown cell mass. In particular, a washing media may be utilized to flush cell culture media from a grown cell mass. For example, a washing media can include Phosphate Buffered Saline (PBS), citric acid, citric acid/potassium dibasic buffers, or other solutions to rinse or wash a grown cell mass.

By contrast, the term “enrichment media” refers to a liquid for enriching cells after cells have grown into a grown cell mass. In particular, enrichment media contains components that may be added to a grown cell mass to improve the nutritional composition or otherwise improve the quality of a grown cell mass for consumption. For example, enrichment media can include nutrients, such as vitamins, amino acids, antioxidants, fats, or other compounds important for a healthy or nutritious diet. In some instances, the enrichment media may not include nutrients required for cellular growth.

As used herein, the term “nutrients” refers to substances used by cells or organisms to survive, grow, or reproduce. Nutrients can also refer to substances required for animal cells or organisms to survive, grow, or reproduce. For example, nutrients can include vitamins, minerals, amino acids, antioxidants, or fats added to cells necessary for cells, organisms, or humans to survive, grow, and reproduce.

As used herein, the term “gradient washing media” refers to a liquid having a gradient of washing media and enrichment media. In particular, a gradient washing media can refer to a liquid comprising a dynamic gradient that changes to decrease concentration of a washing media and increase a concentration of an enrichment media. For example, a gradient washing media can refer to a liquid comprising 100% washing media at a first time, decreasing concentrations of the washing media and increasing concentrations of enrichment media during intermediate times, and a liquid comprising 100% enrichment media at a later time.

As used herein, the term “culture tank” refers to a vessel used for culturing cells. In particular, a culture tank refers to an environment that can hold cells and cell culture media. For example, a culture tank can include a bioreactor system.

As further used herein, the term “separator” refers to an apparatus or method for separating materials. In particular, a separator refers to an apparatus for separating a grown cell mass from liquids or gels including cell culture media, washing media, enrichment media, and other media. For example, a separator can include a centrifuge, filter, sieve, or coagulant used for sedimentation.

As used herein, the term “washing tank” refers to a vessel for washing cells. In particular, a washing tank refers to a vessel that holds a grown cell mass and at least one of washing media or enrichment media. For example, a washing tank can comprise a tank with one or more openings for adding and removing washing or enrichment media.

As further used herein, the term “exchange media” refers to a liquid or gel comprising compounds that stimulate diffusion of substances into and/or out of cells. In particular, exchange media may comprise a varying concentration of membrane-permeable solutes. When cells are exposed to the exchange media, membrane-permeable solutes can cross the cell membranes from areas of high concentration to areas of low concentration. For example, an exchange media may comprise a high concentration of nutrients to diffuse into intracellular spaces of cells. In another example, an exchange media comprises a low concentration of solutes to cause substances from within the cell to exit the cell.

As further used herein, the term “hypotonic” refers to a property of a solution having a comparatively lower concentration of solutes relative to the concentration of solutes in another solution or cells. In particular, a hypotonic solution comprises a lower concentration of membrane-permeable solutes relative to another solution. In some examples, a hypotonic solution contains a lower concentration of membrane-permeable solutes than is found within an exchange media or cell culture media. For example, a first exchange media may comprise a hypotonic solution that stimulates membrane-permeable solutes to exit a cell.

As further used herein, the term “hypertonic” refers to a property of a solution having a comparatively higher concentration of solutes relative to the concentration of solutes in another solution or cells. In particular, a hypertonic solution comprises a higher concentration of membrane-permeable solutes relative to cell culture media or an exchange media. For example, a hypertonic exchange media can comprise a relatively high concentration of membrane-permeable solutes that enter intracellular spaces of cells within a cell mass.

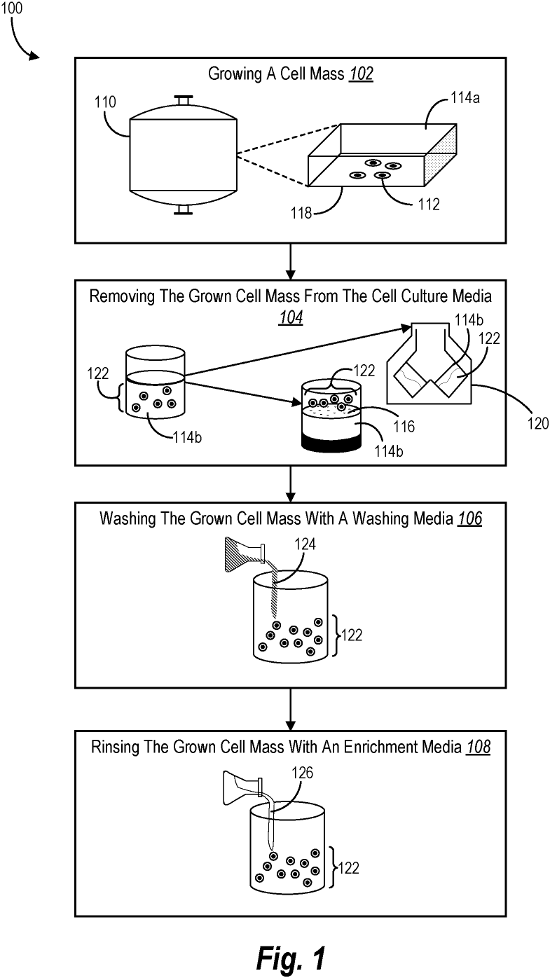

Additional detail will now be provided regarding a disclosed method in relation to illustrative figures portraying example embodiments and implementations of the disclosed method. For example, FIG. 1 illustrates an overview of acts in the disclosed method to wash cells in a washing media and rinse cells in an enrichment media in accordance with one or more embodiments. In particular, FIG. 1 illustrates a series of acts 100 comprising an act 102 of growing a cell mass, an act 104 of removing the grown cell mass from the cell culture media, an act 106 of washing the grown cell mass with a washing media, and an act 108 of rinsing the grown cell mass with an enrichment media.

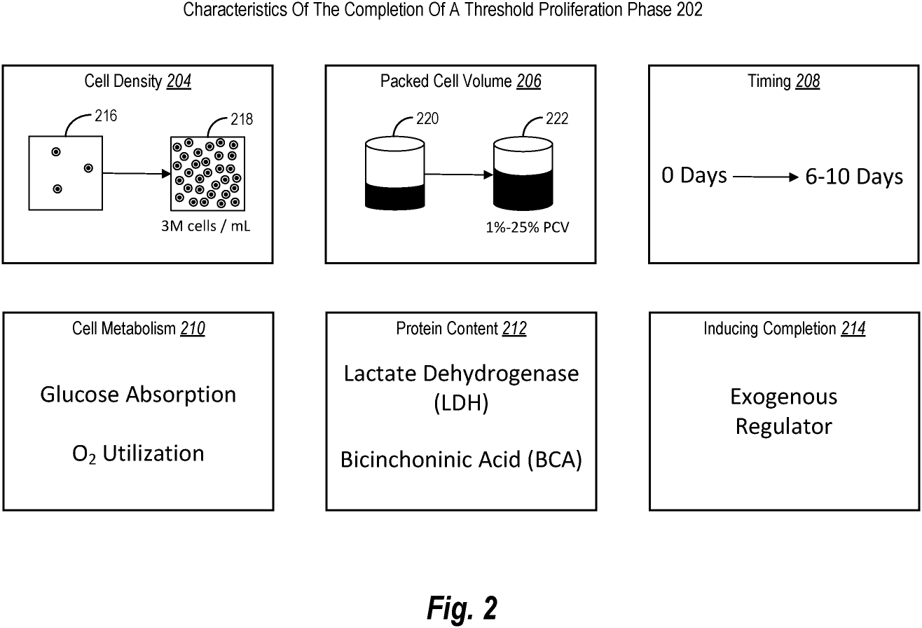

As illustrated in FIG. 1, the series of acts 100 includes the act 102 of growing a cell mass. In some cases, the act 102 comprises growing cells 112 in a cell culture media 114a. For instance, in some examples, the cells 112 are grown in a bioreactor 110. The cells 112 can comprise suspension cells suspended in the cell culture media 114a. Additionally, or alternatively, the cells 112 comprise adherent cells attached to a substrate 118. In some embodiments, the act 102 comprises growing the cells until they have completed a threshold growth or threshold proliferation phase. FIG. 2 and the corresponding paragraphs detail characteristics of cells that have completed a threshold growth phase in accordance with one or more embodiments.

In one example, the act 102 of growing a cell mass comprises filling the bioreactor 110 to 20% volume with cells and cell culture media. To illustrate, if the bioreactor 110 is a 1 kiloliter tank, the act 102 comprises adding 200 L of cells and cell culture media. The disclosed method can comprise a relatively high concentration of cells at the beginning of the growth period (e.g., 15-30%). The cells are allowed to grow and reach exponential growth. After exponential growth is achieved, the disclosed method comprises filling the bioreactor 110 to 100% of its volume with cell culture media. In other embodiments, the disclosed method comprises filling the bioreactor 110 with a different volume cells and cell culture media.

The series of acts 100 further comprises the act 104 of removing the grown cell mass from the cell culture media. In particular, the act 104 comprises separating grown cells 122 from cell culture media 114b. The grown cells 122 may comprise cells from a grown cell mass that are either collected together or disperse throughout an environment. As illustrated, the disclosed method can include the utilization of a centrifuge 120 or a filter 116. For example, the act 104 can comprise using the centrifuge 120 to isolate the grown cells 122 from the cell culture media 114b. In one or more embodiments, removing the cell mass from the cell culture media by centrifugation is the preferred method for separating suspension cells from cell culture media. Because suspension cells are grown in solution, they often require more centrifugation to separate the additional liquid. Therefore, the disclosed method may include added centrifugation time or cycles based on the grown cells being suspension cells. Similarly, samples of agglomerates, or cells grown as partial sheets of tissue in suspension, often include higher proportions of cell culture media relative to samples of adherent cells. Thus, the disclosed method may include added centrifugation time or cycles based on the cells being agglomerates.

As depicted, the centrifuge 120 can comprise a continuous centrifuge or a batch centrifuge. In continuous flow centrifugation, large volumes of material are centrifuged without needing to fill and decant large numbers of centrifuge tubes as is the case in batch centrifugation. When using a centrifuge, in some cases, the mixture of the grown cell and cell culture media constantly flows into the rotor, which runs at a chosen operating speed. Cells sediment out of the flowing stream that carries cell culture media away from the cells. In a preferred embodiment, the centrifuge is operated at a speed where the risk of cell lysis and cell death is low, whereby the cells remain primarily fully intact after they are separated from the cell culture media.

In contrast to continuous centrifugation, in some cases, batch centrifugation may have the advantage of forming cell pellet having relatively higher cell concentrations. For example, batch centrifugation may result in a packed cell volume of nearly 100% without (or with little remaining) cell culture media. However, batch centrifugation is inefficient relative to continuous centrifugation in that it can consume more time.

In some embodiments, the act 104 of removing the grown cell mass from the cell culture media can comprise utilizing the filter 116 to separate the grown cells 122 from the cell culture media 114b. In particular, the filter 116 is a semi-permeable membrane that acts as a barrier that retains the grown cells 122 while allowing the cell culture media 114b to pass through. In some embodiments, the disclosed method uses membrane filtration for separating suspension cells from cell culture media.

In some embodiments, the act 104 of removing the grown cell mass from the cell culture media is replaced with an act of diluting or displacing the cell culture media with washing media. For instance, the grown cell mass may be simply flooded with washing media to dilute and/or displace the cell culture media. This may beneficially reduce the number of separation (e.g. by centrifugation) steps by combining the separation of the cell culture media and the washing media into a single step.

In addition or as an embodiment of a filter, the act 104 can comprise the use of sieving and/or sedimentation. Sieving is like filtration in that cells that are too big to pass through holes in a sieve are retained. The disclosed method can include the use of inline and/or shake sieves. The disclosed method can further include several layers of sieves to separate tissue and microparticles.

In addition, or in the alternative to filtering, the act 104 may further comprise flocculation or sedimentation. Flocculation or sedimentation may be the preferred method to separate agglomerates or clusters of cells from cell culture media. In flocculation/sedimentation, a coagulant is added to collect cells into aggregates. For example, the disclosed method can include adding polymers, multivalent cations, metal salts, or other compounds to cause the grown cells 122 to aggregate. After or while aggregating the cells, the disclosed method can drain or filter out the cell culture media.

In one or more embodiments, the disclosed method utilizes one or more of the above-mentioned methods for removing the cell mass from the cell culture media. For example, the disclosed method can perform the act 104 by utilizing a combination of flocculation, centrifugation, and/or filtering to isolate the grown cells 122 from the cell culture media.

In some embodiments, the act 104 is optional and the disclosed method does not include the act of removing the cell mass from the cell culture media. For example, in some embodiments, the grown cell mass comprises tissue or cells grown as a sheet. Tissue can comprise adherent cells attached to a substrate during growth. In such cases, after the proliferation phase, the cell culture media may be simply drained from the tank leaving the cell tissue attached to the substrate in a relatively dry form that is then ready for washing. Thus, the disclosed method can proceed from the act 102 of growing a cell mass to the act 106 of washing the grown cell mass with a washing media. In such embodiments, the disclosed method may include additional agitation (e.g., mixing or inverting) of the grown cell mass and the washing media to ensure that the cell tissue does not form pockets that hold captured cell culture media.

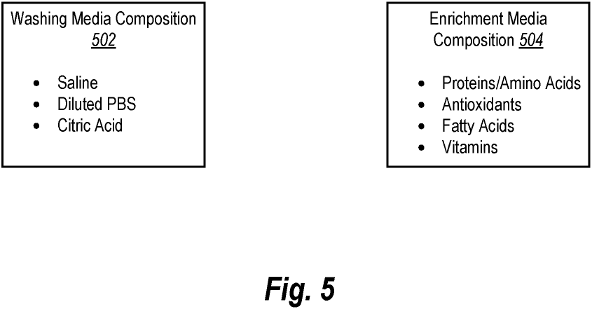

As depicted in FIG. 1, the series of acts 100 further comprises the act 106 of washing the grown cell mass with a washing media. In particular, a washing media 124 is flowed or sprayed across the grown cells 122. The washing media 124 displaces or removes remaining media of the cell culture media 114b from the grown cells 122. Example compositions of the washing media 124 are described below with respect to FIG. 5.

In some embodiments, the act 106 includes pouring the washing media 124 onto the grown cells 122 and agitating the washing media 124 and the grown cells 122. In some examples, the washing media 124 and the grown cells 122 are agitated by gently swirling a centrifuge or container that hold the grown cells 122 and the washing media 124. In some embodiments, the washing media 124 and the grown cells 122 are agitated by utilizing a mixing mechanism. Example mixing mechanisms can include a motorized agitator or an impeller. In some embodiments, the disclosed method includes using a cell retention filter to hold the grown cells 122 while they are being washed with the washing media 124.

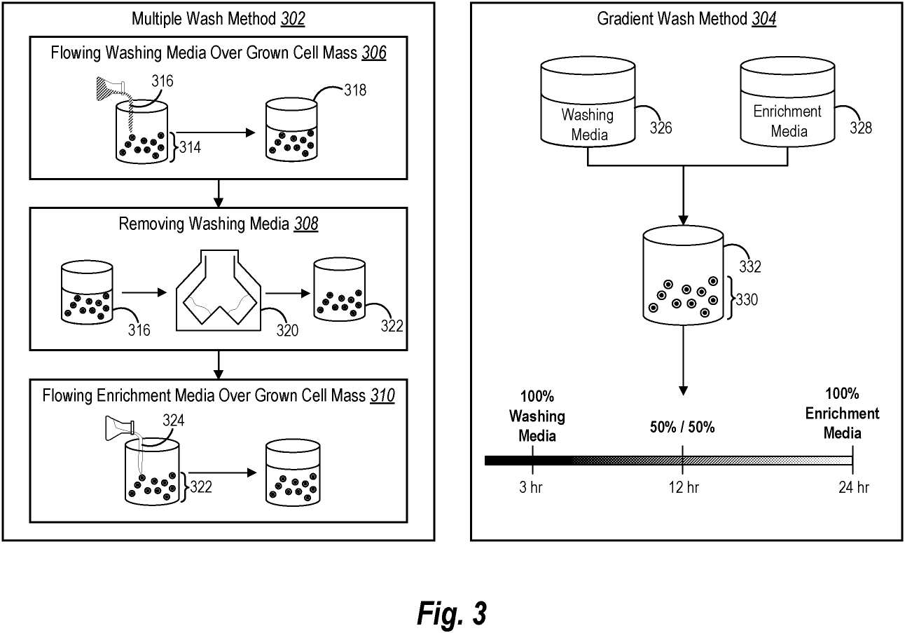

As indicated above, the act 106 comprises various washing methods. For instance, the act 106 can comprise a multiple wash method or a gradient wash method. FIG. 3 and the corresponding paragraph describe an example multiple wash method and an example gradient wash method in accordance with one or more embodiments.

As further illustrated in FIG. 1, the series of acts 100 includes the act 108 of rinsing the grown cell mass with an enrichment media. As depicted, the act 108 comprises washing the grown cells 122 with an enrichment media 126. The enrichment media 126 can reduce the salinity of the grown cells 122. The enrichment media 126 may also act as a final treatment to enrich the grown cells 122. To enrich the grown cells 122, for instance, the enrichment media 126 can include nutrients such as vitamins, amino acids, antioxidants, or fats. The nutrients improve the nutritional composition of the grown cells 122 as a comestible food product. Example compositions of the enrichment media 126 are described below with respect to FIG. 5.

As described previously, the disclosed method comprises washing the grown cell mass once a threshold proliferation phase has completed. FIG. 2 and the corresponding paragraphs describe example characteristics for the completion of a threshold proliferation phase 202. FIG. 2 illustrates determining a threshold proliferation phase has completed based on various characteristics including cell density 204, packed cell volume 206, timing 208, cell metabolism 210, and protein content 212. Additionally, or alternatively, in some embodiments, the disclosed method comprises inducing completion 214.

As illustrated in FIG. 2, one characteristic that indicates the completion of a threshold proliferation phase is cell density 204. FIG. 2 illustrates a beginning cell density 216 corresponding to when cells are first injected into a bioreactor. As the cells grow and proliferate, they increase in cell density. For example, cells can be considered to have completed the proliferation phase when reaching a target cell density 218. In some embodiments, the target cell density 218 is 2 million cells per milliliter, 3 million cells per milliliter, or some density intermediate to these values. In some embodiments, the cell density 204 characteristic applies to suspension cells and not adherent cells.

Another characteristic that indicates the completion of the threshold proliferation phase is packed cell volume (PCV) 206. Generally, PCV refers to the volume of cells in a sample. FIG. 2 illustrates a beginning PCV 220 and a target PCV 222. The beginning PCV 220 reflects the volume of cells in a sample at the beginning of a growth phase. The target PCV 222 indicates that the cells have proliferated and occupy a greater volume. In some embodiments, the target PCV 222 equals at least 1% PCV. In other embodiments, the target PCV 222 equals any value within a range from 1%-25% PCV. In some embodiments, the packed cell volume 206 characteristic applies to evaluating suspension cells and not adherent cells.

In addition or in the alternative to PCV, in some embodiments, cells are considered to have completed the threshold proliferation phase based on timing 208. For example, the disclosed method may proceed to washing the grown cells after cells have been grown for a growth period. The growth period can comprise any length of time. In some embodiments, for instance, the growth period is between 6 days and 140 days.

As further indicated by FIG. 2, cells may also indicate completion of the threshold proliferation phase by cell metabolism 210. Generally, as cells grow, they collectively consume more glucose and oxygen. During exponential growth, it is expected that glucose and oxygen consumption will also rise exponentially. After the cells exit the threshold growth phase, glucose and oxygen consumption will rise more slowly thereby indicating that growth is slowing down. Accordingly, in some embodiments, the disclosed method comprises analyzing glucose absorption and/or O₂ utilization to determine whether the cells have completed the threshold proliferation phase. In one example, oxygen consumption stabilization, e.g., oxygen consumption that is not changing over time or is minimally changing, indicates that the cells are ready for harvesting and therefore washing and finishing.

Further, in some embodiments, the disclosed method determines the completion of a threshold proliferation phase based on protein content 212. Generally, the concentrations of different proteins in the cell culture media and cellular mixture can indicate the slowing of proliferation. For example, increasing lactate dehydrogenase (LDH) content in cell culture media is indicative of cell death. In particular, LDH is a cytoplasmic enzyme that, upon cell membrane failure, is rapidly released into cell culture media. Thus, an increase in LDH content may signal the completion of the completion of the threshold proliferation phase. In another example, bicinchoninic acid (BCA) can be used to analyze the total level of protein in the cell culture media. In particular, a BCA assay may be utilized to measure the protein content in cell culture media. Peaks and plateaus in protein levels can indicate that the cells have completed the threshold proliferation phase.

The disclosed method can further induce completion of the threshold proliferation phase. As illustrated in FIG. 2, the disclosed method can induce completion 214 by exposing cells to an exogenous regulator. The exogenous regulator may cause or facilitate trans-differentiation of the cells to transition the cells from the threshold proliferation phase to a phase of forming mature muscle fibers, such as myofibers and myosin heavy chains. For example, exogenous regulators can include trans-differentiation factors that slow cellular proliferation and/or stimulate trans -differentiation.

In addition or in the alternative to an exogenous regulator, in another example, the disclosed method includes inducing completion 214 by removing the cells from the cell culture media. Generally, when cells are removed from a cultivator, they stop or slow proliferation because they are removed from cell culture media that provides the nutrients required for sustained growth.

As mentioned previously, the disclosed method comprises washing the grown cell mass with a washing media to flush out remaining cell culture media. Generally, the washing media displaces or removes cell culture media from the grown cell mass. FIG. 3 and the corresponding discussion detail two different methods for washing the grown cell mass in accordance with one or more embodiments. FIG. 3 illustrates a multiple wash method 302 and a gradient wash method 304.

Generally, the multiple wash method 302 comprises washing a grown cell mass using separate and distinct media. By way of overview, in one example, the disclosed method comprises growing cells, collecting a grown cell mass, chilling the cells, removing the cell culture media, and applying a first washing media provides a bulk dilution of cell culture media and includes isotonic saline to limit cell rupture from osmotic stress. The first washing media can be centrifuged or otherwise removed from the grown cell mass. A second enrichment media is used to remove residual washing media while also decreasing salt concentration. The second enrichment media may further be centrifuged or otherwise removed from the grown cell mass. As illustrated in FIG. 3, the multiple wash method 302 comprises an act 306 of flowing washing media over a grown cell mass, an act 308 of removing washing media, and an act 310 of flowing enrichment media over the grown cell mass. Although FIG. 3 illustrates one cycle of flowing washing media over the grown cell mass, in some embodiments, the disclosed method includes several cycles of flowing washing media over the grown cell mass.

The multiple wash method 302 includes the act 306 of flowing washing media over the grown cell mass. As illustrated in FIG. 3, a washing media 316 is added to grown cells 314. The grown cells 314 comprise concentrated cells after being optionally separated by centrifuge or filter from cell culture media. In some embodiments, the grown cells 314 are washed with the washing media 316 at a ratio of cells to washing media of 1:1 to 1:4. For example, if the grown cells 314 are a volume of 40 L, the disclosed method includes adding 40 L to 160 L of the washing media 316. Furthermore, in some embodiments, the washing media 316 is added to the grown cells 314 in a washing tank 318. The washing tank 318 may be pressurized to reduce the risk of contamination.

The amount of washing media 316 added to the grown cells 314 may be optimized to efficiently remove cell culture media remnants from the grown cells 314. For example, if too much of the washing media 316 is added, processing time increases and the materials required to process the grown cells 314 increases. If too little of the washing media 316 is used, the grown cells 314 may not be sufficiently coated with the washing media 316 and residual cell culture media may remain in the grown cells 314.

The washing media 316 can be flowed over or exposed to the grown cells 314 for a washing period. The washing period can last anywhere from 5 minutes to 8 hours. If the washing period is too long and if osmolarity is too high, then the grown cells 314 may burst. If the washing period is too short, then the washing media 316 may not have the time needed to equilibrate and perform necessary ion exchanges.

The disclosed method may also include controlling the temperature of the washing media 316. In particular, the washing media 316 can be between -5 C and 45 C. Warmer temperatures have the benefit of faster mass transfers across cell membranes whereas lower temperatures correspond with better cell survival.

In some embodiments, the act 306 further comprises agitating a mixture of the washing media 316 and the grown cells 314. Generally, agitating the mixture of the washing media 316 and the grown cells 314 maximizes exposure of the grown cells 314 to the washing media 316. The increased exposure enhances transfer and equilibration processes between the grown cells 314 and the washing media 316. To accomplish this, the mixture may be stirred in the washing tank 318. The cells may be agitated, circulated via pump, or homogenized using air or gas. The agitation may be limited in intensity and time to preserve the cells. Too much or too severe of agitation can disturb the grown cells 314 by shear force and result in cellular damage.

As further depicted in FIG. 3, the multiple wash method 302 also includes the act 308 of removing washing media. In particular, the disclosed method comprises separating washed grown cells 322 from the washing media 316. In one or more embodiments, the act 106 utilizes methods used to separate the grown cell mass from the cell culture media to separate the washed grown cells 322 from the washing media 316. For example, the act 308 can comprise utilizing a centrifuge 320, filtration, sieving, or flocculation/sedimentation to separate the washed grown cells 322 from the washing media 316.

As further illustrated in FIG. 3, the multiple wash method 302 includes the act 310 of flowing enrichment media over the grown cell mass. In particular, the disclosed method comprises flowing an enrichment media 324 over the washed grown cells 322. The method for flowing enrichment media over the grown cell mass is similar to the method of flowing the washing media over the grown cell mass. More specifically, the washing procedure, ratio of cells to enrichment media, temperature, concentration, agitation, and timing are the same or similar to those described above in relation to the act 306 of flowing washing media over the grown cell mass.

To illustrate, the enrichment media may be flowed over the grown cell mass and agitated. For example, the grown cell mass and the enrichment media may be stirred, circulated via pump, or homogenized with air/gas. In some embodiments, the grown cell mass is covered with the enrichment media 324 at a ratio of cells to enrichment media of 1:1 to 1:4. In some embodiments, the temperature of the enrichment media 324 is between 1 C and 40 C. More specifically, the enrichment media 324 may be at a temperature of 4 C. The disclosed method can further comprise exposing the washed grown cells 322 to the enrichment media 324 for an enrichment period. The enrichment period may be the same as the washing period or longer or shorter than the washing period. In particular, the enrichment period may be 5 minutes to 8 hours.

In some embodiments, the multiple wash method 302 further comprises an additional act of removing the enrichment media. In particular, the enriched washed grown cells can be separated from the enrichment media 324 to remove excess enrichment media. In some examples, the disclosed method comprises centrifuging the mixture of the grown cell mass and the enrichment media 324. Additionally, or alternatively, the cells may be flocculated.

Furthermore, the multiple wash method 302 may include the additional act of drying the grown cell mass. Generally, the grown cell mass may be dried to facilitate a transition of the grown cell mass into a cell-based-meat product ready for consumption by, for example, improving the texture of the cell-based-meat product. For example, the grown cell mass may be dried to have a moisture content between 50% and 80% to mimic the moisture content of conventional meat. For example, the grown cell mass may be dried under a vacuum, spray, or forced drying system.

In the alternative to the multiple wash method 302, the disclosed method can wash the grown cell mass using the gradient wash method. Generally, a gradient washing media is flowed over the grown cell mass. The composition of the gradient washing media changes over time. For example, a washing media can be flowed over the grown cell mass for a set time and gradually transition to the enrichment media.

FIG. 3 illustrates the gradient wash method 304 in accordance with one or more embodiments. By way of overview, the gradient wash method 304 may comprise growing cells, collecting the grown cell mass, centrifuging or otherwise removing the cell culture media from the grown cell mass, chilling the cells, and washing the cells with a gradient wash media. The gradient wash method 304 may utilize two tanks—one holding a washing media 326 and the other holding an enrichment media 328. The two tanks are each connected to a washing tank 332 storing a gown cell mass 330. The concentration of the gradient wash media may start as 100% of the washing media 326 and slowly include more of the enrichment media 328. The gradient wash media continues transitioning until it is 100% (or nearly 100%) composed of the enrichment media 328. As the gradient wash media changes over time, the disclosed method may include draining media from the washing tank 332. For example, the washing tank 332 may drain media at the same rate as the media is being flowed into the washing tank 332. Benefits of the gradient wash method 304 relative to the multiple wash method 302 include improved efficiency by removing the need for removing the washing media.

The disclosed method can include adjusting the time that the washing media 326 and/or the enrichment media 328 are exposed to the gown cell mass 330. For example, and as illustrated in FIG. 3, the gradient wash method 304 is performed within a 24-hour time period. In the first 3 hours (or any time within the optimal washing period of 5 minutes to 8 hours), the gradient washing media features an inflow that is 100% (or nearly 100%) made up of the washing media 326. Over an intermediate period from hour 3 to hour 21, the gradient washing media gradually transitions from an inflow having greater concentrations of the washing media 326 to an inflow having greater concentrations of the enrichment media 328. In the last 3 hours (or any time within an optimal enrichment period of 5 minutes to 8 hours), the gradient washing media inflow is 100% (or nearly 100%) made up of the enrichment media 328. In alternative embodiments, the inflow transitions from less than 100% washing media (e.g., a starting ratio of 95% washing media and 5% enrichment media) to less than 100% enrichment media (e.g. an ending ratio of 5% washing media and 95% enrichment media).

In one example of the gradient wash method 304, the washing media 326 comprises a high osmotic solution containing high concentrations of salt, sugar, etc. The washing media 326 can further include citrate to remove ions from the gown cell mass 330. Additionally, the washing media 326 can further change the pH of the gown cell mass 330. As suggested above, in some cases, the gradient wash method 304 further comprises gradually changing to the enrichment media 328 having a lower salt concentration, little to no citrate, and additional nutrients such as B vitamins. In some embodiments, the disclosed method excludes, from the washing media 326 and the enrichment media 328, acids (such as citrate) that risk denaturation of proteins and result in a mushy texture.

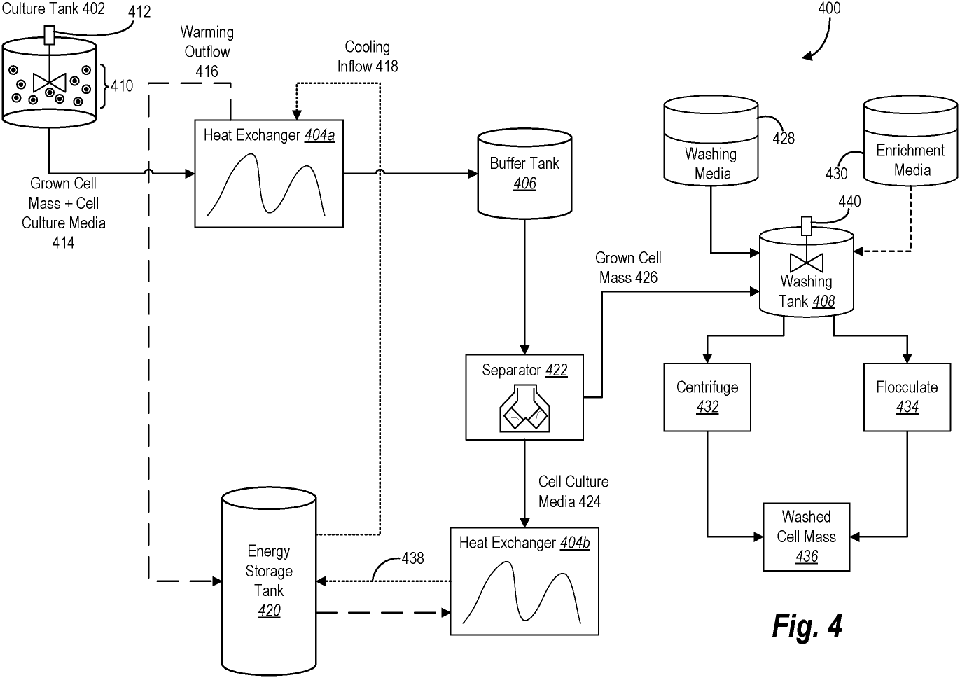

FIG. 4 illustrates an example system for performing the disclosed method in accordance with one or more embodiments. Generally, FIG. 4 includes various components that may be part of a system for washing a grown cell mass. For example, FIG. 4 illustrates a system 400 including culture tank 402, a buffer tank 406, a washing tank 408, and various other components. The disclosed method can include the use of more or fewer components than those illustrated in FIG. 4. Furthermore, in some embodiments, the components in FIG. 4 are organized in a different order.

As illustrated in FIG. 4, the system 400 comprises the culture tank 402. The culture tank 402 provides an environment in which cells can proliferate and complete a threshold growth phase to become a grown cell mass. More particularly, the culture tank 402 contains cell culture media that has nutrients to fuel cell growth. In some embodiments, the culture tank 402 comprises a bioreactor system. Cells 410 in the culture tank 402 are kept at a growth temperature. For example, the cell culture media and the cells 410 can be kept at 40 C for mammalian cells and at a lower temperature for aquatic species. In some embodiments, the culture tank 402 also includes an agitator 412. The agitator 412 mixes or otherwise agitates cells to increase exposure of the cells 410 to the cell culture media.

As further illustrated in FIG. 4, grown cell mass and cell culture media 414 can be harvested and transferred to a buffer tank 406. In some examples, a portion of the grown cell mass is harvested or drained from the culture tank 402 and another portion is left in the culture tank 402 for the next batch of growth. For instance, 80% of the grown cell mass can be extracted from the culture tank 402 for washing, which leaves 20% of the grown cell mass in the culture tank 402 (e.g., for a subsequent growth and proliferation phase).

As mentioned, the grown cell mass and cell culture media 414 are transferred to the buffer tank 406 as an intermediate tank to store grown cells during drainage and before washing. The buffer tank 406 is a sterile vessel that stores cells before separation. The buffer tank 406 may provide the benefit of efficiency. More specifically, in instances where several culture tanks are operating in sync, the culture tanks are drained at regular intervals. In one example where 8 bioreactors are operating in sync, one of them is typically drained every 2 hours. In some embodiments, the buffer tank 406 provides a storage space for grown cells from the culture tanks as downstream processes catch up to the draining schedule.

As further shown in FIG. 4, the system 400 further includes a heat exchanger 404a. In some embodiments, as part of washing the cells, the disclosed method cools the grown cell mass. In the case of mammalian cells, cells are cooled from a growth temperature of 36 C-42C to 1C-20C. In some cases, the grown cell mass is cooled to the lowest temperature possible without killing or freezing the grown cells.

Cooling the cells provides several benefits. For example, cooling cells slows metabolic processes within the cells to slow down lysing and other forms of cell death. Cooled cells are also less active, which extends the amount of time in which additional processing may take place. Furthermore, cooling the grown cell mass increases fluid viscosity and may strengthen cell membranes, which enables the grown cells to better withstand agitation and centrifugal forces in following steps.

The heat exchanger 404a cools the grown cell mass. In the example illustrated by FIG. 4, the heat exchanger 404a cools both the grown cell mass and the cell culture media 414 before the grown cell mass and the cell culture media 414 are separated by a separator 422. Cooling the grown cell mass and the cell culture media 414 together may strengthen cell membranes before they are centrifuged or otherwise separated from the cell culture media. However, cooling both the grown cell mass and the cell culture media 414 requires additional energy as opposed to cooling the lesser volume of only the grown cell mass.

In some embodiments, the heat exchanger 404a cools the grown cell mass after it has been separated from the cell culture media by the separator 422. In such embodiments, the system 400 saves energy because the cell culture media is not cooled. However, because cells in the grown cell mass are not cooled prior to separation, the cells may be more sensitive. Thus, separating the grown cell mass from the cell culture media at growth temperature may require centrifugation at slower speeds to prevent cellular death.

As further illustrated in FIG. 4, the separator 422 removes the grown cell mass 426 from the cell culture media 424 (as described previously regarding act 104 in FIG. 1). The separator 422 comprises a continuous centrifuge that separates the grown cell mass and the cell culture media 414 into a heavy phase and a light phase of centrifugation. The heavy phase contains or collects the grown cell mass 426 and the light phase contains or collects the cell culture media 424. In some embodiments, the continuous centrifuge is operated at a revolutions per minute (RPM) or relative centrifugal force (G) that ensures cell wall structural integrity is maintained during centrifugation. At excessively high RPMs or Gs, the cells may burst. At lower RPMs and Gs, the separation is insufficient, and the heavy phase contains excessive growth media.

As further shown in FIG. 4, the grown cell mass 426 can be transferred to a washing tank 408, and the cell culture media 424 can be flowed to a heat exchanger 404b. The heat exchangers 404a-404b work in conjunction with an energy storage tank 420 to improve energy efficiency of the system 400. Generally, the heat exchangers 404a-404b recuperate energy used to cool the grown cell mass.

As mentioned and as further depicted in FIG. 4, the heat exchanger 404a cools the grown cell mass (and in some embodiments, the cell culture media) from a growth temperature (e.g., 40 C) to a cooled temperature (e.g., 1 C). In some implementations, the cooled temperature comprises any temperature below an optimal growing temperature for cells in the grown cell mass. Cooling the cells in the heat exchanger 404a utilizes a cooling inflow 418 (e.g., 1 C) and results in a warming outflow 416 (e.g., 36C).

To facilitate heating and cooling, the system 400 optionally includes the energy storage tank 420. In particular, the energy storage tank 420 facilitates the efficient exchange of energy between the cell culture media 424 at a cooled temperature and the grown cell mass at the growth temperature. More specifically, in some embodiments, the energy storage tank 420 contains cooled liquid, which sinks to the bottom and a warmed liquid that rises to the top of the energy storage tank 420. In one example, the energy storage tank 420 is a cigar-shaped energy storage tank. As indicated by the different patterns of lines outflowing and inflowing into the energy storage tank 420, the warming outflow 416 warms the liquid, and the cooling outflow 438 from the heat exchanger 404b cools the liquid within the culture tank 402.

As mentioned, the grown cell mass 426 is transferred to the washing tank 408. In the washing tank 408, the system 400 flows, sprays, or immerses washing media 428 and/or enrichment media 430 over the grown cell mass 426. The washing tank 408 can also include an agitator 440 for stirring or otherwise agitating the grown cell mass during the washing process. The agitator 440 agitates the grown cell mass and the washing media or enrichment media. While FIG. 4 illustrates the agitator 440, the cells may be agitated in the washing tank 408, circulated via pump, or homogenized with air/gas.

As further illustrated in FIG. 4, the disclosed method can utilize the multiple wash method described above in the system 400. In particular, the washing media 428 can be flowed over the grown cell mass in the washing tank 408. The washing media is removed from the grown cell mass by centrifuge 432. More specifically, the washing media can be removed using (i) a second centrifuge or (ii) the separator 422 used to separate the cell culture media 424 and the grown cell mass 426.

The disclosed method can also comprise removing the washing media from the grown cell mass by flocculation 434. In flocculation 434, the grown cell mass is collected into aggregates by the addition of multivalent cations, metal salts, or polymers. Flocculation may be more desirable for cell-based meat products where high moisture content is acceptable. Regardless of the removal method, when the washing media has been removed, the system 400 is left with a washed cell mass 436.

As described above, when using the multiple wash method, an enrichment media is added to, flowed over, or immerses the washed cell mass 436. In some embodiments, the enrichment media is added to the washed cell mass 436 in the washing tank 408. In other embodiments, the enrichment media is added to the washed cell mass 436 in a separate enrichment tank.

In addition to the multiple wash method, FIG. 4 also illustrates the gradient wash method described above in the system 400. In particular, the washing tank 408 is connected to both the washing media 428 and an enrichment media 430. As described above, the disclosed method can comprise flowing (or immersing with) a gradient washing media by changing concentrations of the washing media 428 and the enrichment media 430 over time. After the gradient washing media has been flowed over the grown cell mass in the washing tank 408, the cells can be formed into a final cell-based-meat product.

As mentioned, the washing media and the enrichment media remove undesirable aromas, tastes, and textures while also improving the nutritional composition of cell-based-meat products. FIG. 5 provides an overview of compositions of the washing media and the enrichment media to provide texture, flavor, aromatic, and nutritional benefits in accordance with one or more embodiments.

As indicated above, the washing media displaces/removes cell culture media, washes the grown cell mass, and/or reduces cell lysis. The enrichment media enriches the grown cell mass with nutrients, preservatives (e.g., antioxidants), flavors, aromas, colors, and/or texturizers. But neither the washing media nor the enrichment media are cell culture media. For instance, in some embodiments, neither the washing media nor the enrichment media contain growth factors that cause the cells to grow—growth factors that the cell culture media can include. FIG. 5 illustrates an example of washing media composition 502 and an example of enrichment media composition 504. In one such example of the washing media composition 502, the washing media is diluted phosphate buffered saline (PBS) and citric acid. Citric acid may beneficially lower cholesterol. In another example, the washing media is citric acid/potassium dibasic buffers. Washing media made of isotonic saline also has osmolarity that limits cell rupture and provides a cost-effective bulk dilution.

By contrast, in examples of the enrichment media composition 504, the enrichment media comprises a hypotonic saline with low salt content. The enrichment media can also contain compounds that will be taken up by the grown cell mass. In particular, the enrichment media can contain proteins/amino acids, antioxidants, fats, fatty acids, oils, and vitamins. In some embodiments, the enrichment media contains comestible compounds. The enrichment media can also contain antioxidants such as B-vitamins, butylated hydroxyanisole (BHA), butylated hydroxytoluene (BHT), or other natural extracts from plants with antioxidant properties such as cherry, alpha tocopherols, and other extracts. These antioxidants prevent the oxidation of fats during the drying process.

Furthermore, in some embodiments, the grown cell mass is treated to improve cell uptake of nutrients from an enrichment media. For example, the cell mass may be heat shocked or electroporated, whereby mass transfer through the cell wall is increased. The disclosed method can subsequently rinse the grown cell mass with enrichment media after heat shock or electroporation.

The washing media and/or the enrichment media can contain materials that control sensory aspects of a cell-based-meat product. Sensory aspects include aroma, taste, appearance, mouthfeel, and texture. In particular, the washing media and/or the enrichment media can facilitate the removal of volatile substances associated with unsuitable aromas. For example, many lipid oxidation products are volatile and result in the formation of primary and secondary compounds including aldehydes, ketones, and alcohols that impart unsuitable aromas and unsuitable flavors in a cell-based meat product that are uncharacteristic of slaughtered meat. More specifically, potentially undesirable cell culture media components and degradants may include tyrosine, phenylalanine, tryptophan, threonine, methionine, isoleucine, choline, ammonium ions, and others. In particular, methionine when not broken down can impart a boiled cabbage like smell and flavor to the final product. Isoleucine smells and tastes like cookies when not broken down. Cysteine, when not broken down, can impart a rotten egg-like smell and flavor to the final product. Ammonia in the medium can impart a urine or sweat-like smell. The washing and/or enrichment media can also remove iron and iron complexes, which may be a source of off aromas.

Furthermore, the addition of B-vitamin antioxidants in enrichment media minimize oxidative pathways leading to off aromas. In particular, lipid peroxidation deteriorates conventional meat and meat products and imparts undesirable odors and texture to the meat products. Typically, polyunsaturated fatty acids such as linoleic acid undergo peroxidation by reactive oxygen species (ROS). The addition of B-vitamins in the enrichment media quench ROS and thereby prevent the peroxidation of lipids.

As mentioned, additives in the washing media and/or enrichment media may also improve the taste of a cell-based-meat product. In particular, the washing media removes undesirable cell culture media components and degradants that impart off flavors to cell-based meat products. In one example, methionine, isoleucine, cysteine, and ammonia are all components of cell culture media that may be washed from a grown cell mass using the washing media. Off flavors may be metallic in nature and result from the particular species of iron present in the grown cells. Metallic components may be removed by components in the washing media that bind iron or replace the present iron species (e.g., iron-binding protein) with a different iron species having a more favorable flavor and/or aroma. As mentioned, B-vitamin antioxidants in washing media and enrichment media can minimize oxidative pathways leading to unsuitable flavors.

Components of the washing media and/or enrichment media also improve the appearance of a final cell-based-meat product. For example, color may be introduced by components with color-promoting or color inhibiting properties. Examples of such components include beet juice, carrot juice, other plant-based juices, hydrolysates or other peptones, and other coloring agents. Color may be retained to prevent graying by adding iron or by preventing oxidation through the washing and enriching steps. Furthermore, color may be removed by components that have chelating properties. Examples of such components include sodium cluconate, citrate, ethylenediaminetetraacetic acid (EDTA), and other compounds.

The washing media and/or the enrichment media may also include components that improve the texture of a cell-based-meat product. In particular, the enrichment media can include enzymes, such as cross linkers, to improve the texture. For instance, enrichment media can include transglutaminase for cross linking. Furthermore, the washing media and the enrichment media can include a balanced concentration of NaCl salt buffers that stabilize proteins for a meatier texture.

Aside from controlling sensory aspects of a cell-based-meat product, the washing media and the enrichment media may be used to control osmolarity. Generally, the cell growth media is very high in osmolarity (e.g., cells often grow well in salty conditions) and choosing the proper osmolarity for the washing media and the enrichment media must be made with various considerations. If the media have higher osmolarity than the cells, then media components may be taken up by the cells quicker. However, it may be necessary to first reduce the osmolarity of the cells with the washing media (e.g., to a more palatable salt content) to later utilize an enrichment media that relies on high osmolarity to increase cell uptake. Generally, the disclosed method may comprise adjusting salts, including sodium chloride (NaCl), potassium (K), calcium (Ca), magnesium (Mg), iron (Fe), and other elements, to hit a target threshold of osmolarity to facilitate nutrient uptake.

The washing media and the enrichment media may also be used to alter chemistry within the grown cell mass. In particular, the isoelectric point (pl) of conventional meat proteins tends to be around 5-5.5. The disclosed method can comprise altering the pl based on the pH (or vice versa) to adjust behaviors of the grown cell mass such as ionic retention and moisture content. More specifically, if the pH is lower than the pl, then the proteins will have an overall positive net charge and (R-NH₃+ and R-COOH) groups will be present in abundance. If the pH is greater than the pl, then the proteins will have an overall negative charge and (R-NH₂ and COO) groups will be present in abundance. If the pH equals the pl, then the proteins of the grown cell mass will have an overall neutral charge.

Furthermore, the disclosed method can include optimizing salt concentration within the washing media and/or the enrichment media. The salt concentrations may be chosen for optimal protein stability. Salt concentrations in a suitable range will stabilize proteins and also contribute to a more desirable meatier texture. For example, salt content of the media may be altered by increasing the amount of sodium cation (Na+) and/or chloride anion (Cl-) in the washing media and/or the enrichment media. The salt content in the washing media and the enrichment media can be ratioed to achieve the desired ratio in the final cell-based-meat product. For example, methods for reaching a suitable salt concentration include a concentration of Na+ or Cl— in the washing and/or enrichment media that would result in Na+ or Cl— values in the final product that are within the range of conventional meat or at least leading to a more positive sensory experience. To increase exposure to maximize absorption of NaCl into the cells, a higher washing media/enrichment media-to-cell ratio can be used. Additionally, agitation can increase absorption.

Additionally, potassium content of the washing media and/or enrichment media can be altered by increasing or decreasing the amount of potassium cation (K+) in the media. Like the methods for adjusting Na+ and Cl— content in the cell-based-meat product. In particular, cations in the washing media and the enrichment media are ratioed to achieve the desired ratio in the final product. Again, to increase exposure to maximize absorption, a higher washing media/enrichment-media-to-cell ratio or agitation may be used.

In some embodiments, the washing media and/or the enrichment media are specified based on species of cell. For example, the media can include chelators, colors, flavors, and antioxidants to maintain different sensory qualities corresponding with beef, chicken, duck, pork, fish, and other species. The enrichment media may also adjust the overall macro-nutrient content (e.g., protein, fat, moisture, carbohydrates, etc.) and pH of the cell-based-meat products to mimic the macro-nutrient content and pH of conventional meats.

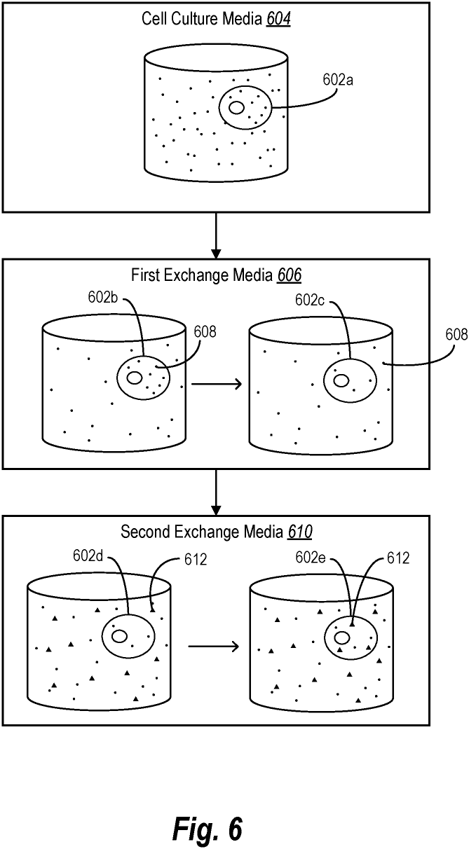

As discussed previously, in some cases, the disclosed method flows a series of media and buffers to improve sensory aspects and nutritional composition of a grown cell mass. In some embodiments, the described methods are used to wash and add nutrients to intercellular spaces within a cell mass. Additionally, in some implementations, the disclosed method uses exchange media to change the concentration of membrane-permeable solutes in intracellular spaces within cells of a cell mass. In accordance with one or more embodiments, FIG. 6 and the corresponding paragraphs describe utilizing exchange media to remove and/or add solutes within cells.

In some examples, the disclosed method comprises flowing exchange media having varying concentrations of solutes and nutrients to stimulate the diffusion of such solutes and nutrients into and/or out of individual cells. In diffusion, particles move from areas of higher concentration to areas of lower concentration until equilibrium is reached. Exchange media with high particle concentrations will cause the cells to take up particles. Exchange media with lower particle concentrations relative to the cell will cause membrane-permeable particles to leave the cell. By using such exchange media, the disclosed method can change concentrations of various solutes and nutrients within the cell. In addition to changing concentrations of particles within the cells, exchange media may also be used to wash particles off the surfaces of cells or adhere particles to the surfaces of the cells.

FIG. 6 illustrates an example series of exchange media in accordance with one or more embodiments of the present disclosure. By way of overview, cells are grown within a cell culture media. The cell culture media is removed, and a first exchange media is added to the cells. In some examples, the first exchange media is hypotonic relative to the cell culture media. More specifically, the first exchange media comprises a lower concentration of solutes relative to the cell culture media. A second exchange media is added to the cells. The second exchange media may be hypertonic and comprise a higher concentration of solutes that diffuse over time into the cell.

As shown in FIG. 6, the cells are grown in cell culture media. For example, a cell 602a represents an adherent cell or a cell grown in suspension. In either case, the cell 602a is grown in contact with cell culture media 604. As described previously, the cell culture media 604 comprises various solutes or components that stimulate cell growth. Some membrane-permeable solutes within the cell culture media 604 enter the cell 602a to reach equilibrium. As mentioned, some of these membrane-permeable solutes stimulate cell growth but also negatively impact sensory aspects or nutrition of the cells. The disclosed method may include steps to remove undesirable membrane-permeable solutes from within the cell 602a.

As further illustrated in FIG. 6, the disclosed method may comprise flushing the cells with a first exchange media 606. In some embodiments, the first exchange media 606 is hypotonic relative to the cell culture media. Accordingly, in some implementations, the first exchange media comprises a lower concentration of solutes relative to the cell culture media. Over time, membrane-permeable solutes move from an intracellular space within the cell 602b to outside the cell 602c to reach equilibrium. As illustrated, the cell 602b contains membrane-permeable solutes 608. In some examples, the membrane-permeable solutes 608 comprise solutes that entered the cell by diffusion during the cellular growth stage. The membrane-permeable solutes 608 may comprise an undesirable cell culture media component or a cell culture media component at an undesirable concentration, such as tyrosine, phenylalanine, tryptophan, threonine, methionine, isoleucine, choline, ammonium ions, salts, and others.

During the wash with the first exchange media 606, at least some of the membrane-permeable solutes 608 leave the cell to reach an equilibrium with the first exchange media 606. As illustrated in FIG. 6, the cell 602c has a lower concentration of the membrane-permeable solutes 608 relative to the cell 602b and the cell culture media 604. In some examples, the first exchange media 606 comprises different components to reduce osmotic stress on the cells.

In some examples, the first exchange media comprises a decreasing solute gradient to reduce osmotic stress on cells within the cell mass. More specifically, the decreasing solute gradient may begin with a solute concentration similar to the solute concentration of the cell culture media 604. The solute concentration is decreased over time to minimize osmotic shock of the cells. The solute concentration is gradually decreased until it reaches a target concentration. In some implementations, the disclosed method comprises flushing the cells with a series of intermediate first exchange media with decreasing solute concentrations.

As further illustrated in FIG. 6, the disclosed method may utilize a second exchange media 610. In some examples, the second exchange media is hypertonic relative to the first exchange media. In other examples, the second exchange media is hypertonic relative to the cell culture media. In some implementations, the second exchange media 610 comprises membrane-permeable solutes including nutrients or other solutes for diffusion into the cell. FIG. 6 illustrates a cell 602d exposed to the second exchange media 610. Over time, nutrients 612 from the second exchange media 610 enter the cell 602e by diffusion. The nutrients 612 may comprise membrane-permeable substances or membrane-impermeable substances. In some examples, the nutrients 612 comprise vitamins, amino acids, antioxidants, proteins, carbohydrates, or fats. Additionally, in some examples, at least a portion of the nutrients 612 adhere to an external surface of the cell 602e.

In some examples, to protect the cells in the cell mass from osmotic stress, the disclosed method comprises flushing the cell mass with the second exchange media 610 by utilizing an increasing solute gradient. In particular, the second exchange media 610 transitions from a solution having a lower concentration of solutes to a solution having a higher concentration of solutes. For example, the solution having the lower concentration of solutes can have a solute concentration similar to a solute concentration of a previous solution (e.g., the first exchange media 606 or the cell culture media 604). A series of second exchange media with increasing solute concentrations is added until a target second solute concentration. In some examples, the disclosed method flushes the cell mass with a series of intermediate second exchange media with progressively higher solute concentrations.

The disclosed method may utilize different methods to determine when to stop flowing exchange media over the cells. For example, the disclosed method may flow the first exchange media (and/or a decreasing solute gradient) over the cell mass for a flushing time period. The first exchange media is removed after the flushing time period and the second exchange media is added to the cells. The second exchange media (and/or an increasing solute gradient) may be flowed over the cells for the same flushing time period. In some examples, the second exchange media is flowed over the cells for a different second flushing time period.

Additionally, or alternatively, the disclosed method comprises transitioning between exchange media based on effluent composition. In particular, the composition of the effluent is generally indicative of solutes that have passed into or out of the cells. For example, cells initially exposed to a first exchange media may expel a higher volume of solutes into the first exchange media. Effluent from this initial exposure may have a greater concentration of the solutes. The First exchange media may be flowed over the cells until the effluent composition is substantially similar to the composition of the first exchange media. The disclosed method may comprise flowing the first exchange media until the first effluent is substantially similar to the composition of the first exchange media. Similarly, the disclosed method may halt flowing of a second exchange media when the second effluent has a composition substantially similar to the second exchange media.

In some implementations, the disclosed method comprises using a single exchange media. In particular, the exchange media can include both a low concentration of undesirable cell-permeable solutes and a high concentration of nutrients. The low concentration of undesirable cell-permeable solutes causes the solutes to diffuse out of the cells while the high concentration of nutrients simultaneously causes the nutrients to diffuse into the cells. As with the first exchange media and the second exchange media, the disclosed method may utilize a single gradient exchange media that slowly transitions from a composition similar to the cell culture media to a target composition.

In some implementations, the disclosed method comprises flushing the cells with exchange media in addition to utilizing washing media and enrichment media. For example, the disclosed method may comprise flushing cells with washing media, enrichment media, a first exchange media, and a second exchange media. In another implementation, the washing media and the enrichment media comprise the first exchange media and the second exchange media, respectively. More specifically, the washing media may comprise a low concentration of particulates to draw out undesirable solutes from within the cells. The enrichment media may contain a high concentration of membrane-permeable nutrients that enter the cells.

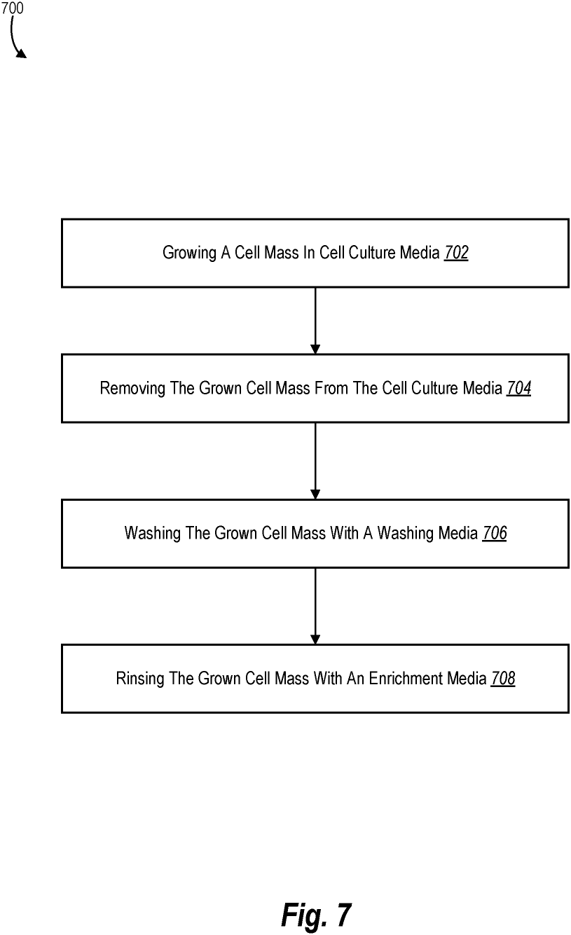

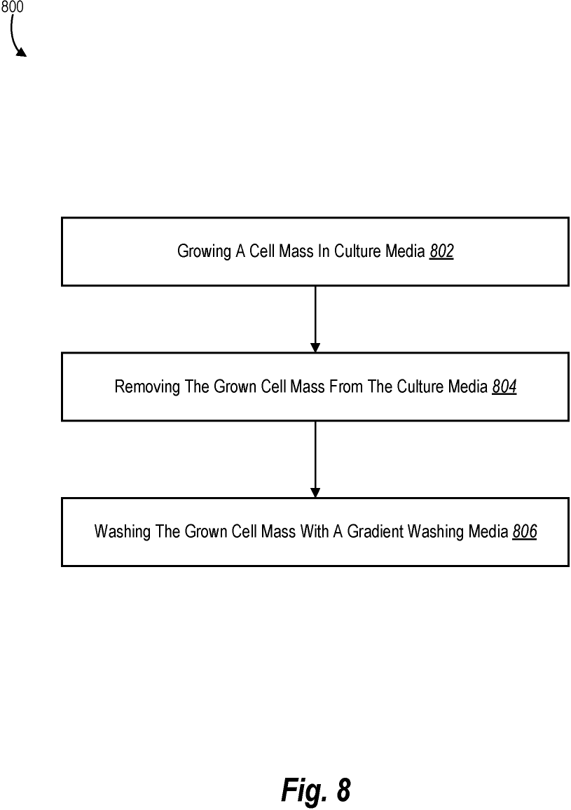

FIGS. 1-6, the corresponding text, and the examples provide several different systems, methods, techniques, components, and/or devices relating to washing and enriching a grown cell mass in accordance with one or more embodiments. In addition to the above description, one or more embodiments can also be described in terms of flowcharts including acts for accomplishing a particular result. FIGS. 7-8 illustrate such flowcharts of acts. The acts described herein may be repeated or performed in parallel with one another or in parallel with different instances of the same or similar acts.

FIG. 7 illustrates a flowchart of a series of acts 700. By way of overview, the series of acts 700 includes an act 702 of growing a cell mass in cell culture media, an act 704 of removing the grown cell mass from the cell culture media, an act 706 of washing the grown cell mass with a washing media, and an act 708 of rinsing the grown cell mass with an enrichment media.

The series of acts 700 includes the act 702 of growing a cell mass in cell culture media.

As illustrated in FIG. 7, the series of acts 700 includes the act 704 of removing the grown cell mass from the cell culture media. In particular, the act 704 comprises removing at least a portion of the grown cell mass from the cell culture media. In some embodiments, the act 704 further comprises removing at least a portion of the grown cell mass from the cell culture media based on the grown cell mass completing a threshold proliferation phase. In some embodiments, the threshold proliferation phase is complete when the grown cell mass reaches at least one of a viable cell density of 3 million cells per milliliter or a packed cell volume of between 1% and 25%. Additionally, in some embodiments, the act 704 further comprises stimulating the completion of the threshold proliferation phase by exposing the grown cell mass to an exogenous regulator.

The series of acts 700 further comprises the act 706 of washing the grown cell mass with a washing media. In particular, the act 706 comprises washing the grown cell mass with a washing media to flush out remaining cell culture media. In some embodiments, the washing media does not comprise growth factors for stimulating the grown cell mass to grow. Additionally, in some embodiments, the act 706 further comprises washing the grown cell mass with the washing media by: agitating both the grown cell mass and the washing media, circulating the grown cell mass and the washing media utilizing a pump, and homogenizing the grown cell mass and the washing media utilizing gas. In some embodiments, washing the grown cell mass with the washing media comprises flowing the washing media over the grown cell mass and flowing intermediate media comprising increasing concentrations of the enrichment media over time. In some embodiments, washing the grown cell mass further comprises flowing the washing media over the grown cell mass and agitating both the washing media and the grown cell mass. In some embodiments, the washing media is at a temperature between and including -5 C and 45 C.

The series of acts 700 illustrated in FIG. 7 further includes the act 708 of rinsing the grown cell mass with an enrichment media. In particular, the act 708 comprises rinsing the grown cell mass with an enrichment media comprising nutrients. In some embodiments, the enrichment media does not comprise growth factors for stimulating the grown cell mass to grow.

In some embodiments, the series of acts 700 includes an additional act of drying the rinsed grown cell mass.

FIG. 8 illustrates a series of acts 800. By way of overview, the series of acts 800 includes an act 802 of growing a cell mass in culture media, an act 804 of removing the grown cell mass from the culture media, and an act 806 of washing the grown cell mass with a gradient washing media.

The series of acts 800 includes the act 802 of growing a cell mass in culture media. In particular, the act 802 comprises growing a cell mass in cell culture media.

As illustrated in FIG. 8, the series of acts 800 includes the act 804 of removing the grown cell mass from the culture media. The act 804 comprises removing the grown cell mass from the cell culture media. In some embodiments, removing the grown cell mass from the cell culture media comprises separating the grown cell mass from the cell culture media via centrifugation.

As further illustrated in FIG. 8, the series of acts 800 includes the act 806 of washing the grown cell mass with a gradient washing media. The act 806 comprises washing the grown cell mass with a gradient washing media by decreasing concentrations of washing media and increasing concentrations of enrichment media over time. In some embodiments, the enrichment media comprises nutrients comprising at least one of vitamins, amino acids, antioxidants, or fats. Furthermore, in some embodiments, the nutrients comprise macro-nutrients comprising at least one of protein, fat, moisture, or carbohydrates. In some embodiments, the enrichment media changes a pH of the grown cell mass.

In some embodiments, the series of acts 800 further comprises cooling the grown cell mass before or after removing the grown cell mass from the cell culture media.

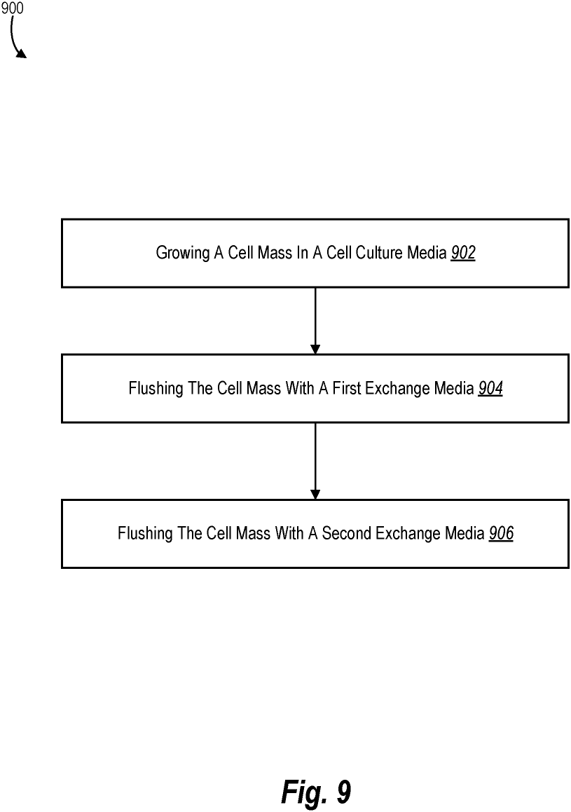

FIG. 9 illustrates a series of acts 900 for flushing a cell mass with a first and a second exchange media in accordance with one or more embodiments. In particular, the series of acts 900 includes an act 902 of growing a cell mass in a cell culture media.

FIG. 9 further illustrates an act 904 of flushing the cell mass with a first exchange media. In some embodiments, flushing the cell mass with the first exchange media causes a first set of membrane-permeable solutes to diffuse out of intracellular spaces of cells in the cell mass. In some examples, the first exchange media is hypotonic relative to the cell culture media. In some implementations, the first exchange media has a lower concentration of membrane-permeable solutes relative to the cell culture media. Furthermore, in some examples, the first exchange media comprises a decreasing solute gradient, and the first exchange media transitions from a solution having a higher concentration of solutes to a solution having a lower concentration of solutes. Additionally, in some cases, flowing the first exchange media comprising the decreasing solute gradient across the cell mass provides a gradual change in solute concentrations, whereby osmotic stress on cells of the cell mass is reduced. In some implementations, the decreasing solute gradient starts at a solute concentration substantially similar to a solute concentration of the cell culture media.

The series of acts 900 includes an act 906 of flushing the cell mass with a second exchange media. In some embodiments, flushing the cell mass with the second exchange media causes a second set of membrane-permeable solutes to diffuse into the intracellular spaces of the cells. In some embodiments, the second exchange media is hypertonic relative to the first exchange media. In some examples, the second exchange media has a higher concentration of membrane-permeable solutes relative to the first exchange media. Furthermore, the second exchange media may comprise an increasing solute gradient, wherein the second exchange media transitions from a solution having a lower concentration of solutes to a solution having a higher concentration of solutes. Additionally, in certain implementations, flowing the second exchange media comprising the increasing solute gradient across the cell mass provides a gradual change in solute concentrations, whereby osmotic stress on cells of the cell mass is reduced. Further, in some cases, the increasing solute gradient starts at a solute concentration substantially similar to a solute concentration of the first exchange media. In certain implementations, the increasing solute gradient includes increasing concentrations of nutrients. In some embodiments, at least a portion of nutrients diffuses into cells of the cell mass, and at least a second portion of the nutrients adheres to an external surface of the cells.

In some implementations, the series of acts 900 includes an act of flushing the cell mass with the first exchange media until a first effluent has a composition substantially similar to the first exchange media. In some examples, the series of acts 900 includes an act of flushing the cell mass with the second exchange media until a second effluent has a composition substantially similar to the second exchange media. Furthermore, in some examples, the series of acts 900 comprises flushing the cell mass for a flushing time period.

In addition, or in the alternative, to series of acts illustrated in FIGS. 7-9, this disclosure includes an additional set of acts. In some implementations, the additional set of acts comprises a method for enriching cultured meat products comprising: growing a cell mass in cell cultured media; removing the grown cell mass from the cell culture media; and washing the grown cell mass with a gradient washing media by decreasing concentrations of washing media and increasing concentrations of enrichment media over time. In some embodiments, the enrichment media comprises nutrients comprising at least one of vitamins, amino acids, antioxidants, proteins, carbohydrates, or fats.

Furthermore, in addition, or in the alternative, to series of acts depicted in FIGS. 7-9, this disclosure includes an apparatus for finishing a cell mass for cultured meat preparation. In some implementations, the apparatus comprises a culture tank for growing the cell mass in cell culture media; a separator for removing a grown cell mass from at least one of the cell culture media, a washing media, or an enrichment media; and a washing tank for storing the grown cell mass and flowing at least one of the washing media or the enrichment media over the grown cell mass. In some implementations, the separator comprises at least one of a centrifuge or a filter. In some examples, the apparatus further comprises a heat exchanger for cooling the grown cell mass below its optimal growing temperature. In some embodiments, the apparatus further comprises an agitator for agitating the grown cell mass and at least one of the cell culture media, the washing media, or the enrichment media.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

The present invention may be embodied in other specific forms without departing from its spirit or essential characteristics. Indeed, the described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A method for enriching cultured meat products, the method comprising:
growing a cell mass in cell culture media;
removing at least a portion of the grown cell mass from the cell culture media;
washing the grown cell mass with a washing media to flush out remaining cell culture media; and
rinsing the grown cell mass with an enrichment media comprising nutrients.

2. The method of claim 1, further comprising washing the grown cell mass with the washing media by:
agitating both the grown cell mass and the washing media;
circulating the grown cell mass and the washing media utilizing a pump; and
homogenizing the grown cell mass and the washing media utilizing gas.

3. The method of claim 1, wherein washing the grown cell mass with the washing media comprises flowing the washing media over the grown cell mass and flowing intermediate media comprising increasing concentrations of the enrichment media over time.

4. The method of claim 1, further comprising removing at least a portion of the grown cell mass from the cell culture media based on the grown cell mass completing a threshold proliferation phase.

5. The method of claim 4, wherein the threshold proliferation phase is complete when the grown cell mass reaches at least one of a viable cell density of 3 million cells per milliliter or a packed cell volume of between 1% and 25%.

6. The method of claim 1, wherein the washing media is at a temperature between and including -5 C and 45 C.

7. A method for enriching cultured meat products, the method comprising:
growing a cell mass in cell culture media;
removing the grown cell mass from the cell culture media; and
washing the grown cell mass with a gradient washing media by decreasing concentrations of washing media and increasing concentrations of enrichment media over time.

8. A method for enriching cell based comestible food products, the method comprising:
growing a cell mass in a cell culture media;
flushing the cell mass with a first exchange media; and
flushing the cell mass with a second exchange media.

9. The method of claim 8, wherein:
flushing the cell mass with the first exchange media causes a first set of membrane-permeable solutes to diffuse out of intracellular spaces of cells in the cell mass; and
flushing the cell mass with the second exchange media causes a second set of membrane-permeable solutes to diffuse into the intracellular spaces of the cells.

10. The method of claim 8, wherein the first exchange media is hypotonic relative to the cell culture media.

11. The method of claim 10, wherein the first exchange media comprises a decreasing solute gradient and wherein the first exchange media transitions from a solution having a higher concentration of solutes to a solution having a lower concentration of solutes.

12. The method of claim 11, wherein flowing the first exchange media comprising the decreasing solute gradient across the cell mass provides a gradual change in solute concentrations, whereby osmotic stress on cells of the cell mass is reduced.

13. The method of claim 11, wherein the decreasing solute gradient starts at a solute concentration substantially similar to a solute concentration of the cell culture media.

14. The method of claim 8, wherein the second exchange media is hypertonic relative to the first exchange media.

15. The method of claim 8, wherein the second exchange media comprises an increasing solute gradient and wherein the second exchange media transitions from a solution having a lower concentration of solutes to a solution having a higher concentration of solutes.

16. The method of claim 15, wherein flowing the second exchange media comprising the increasing solute gradient across the cell mass provides a gradual change in solute concentrations, whereby osmotic stress on cells of the cell mass is reduced.

17. The method of claim 15, wherein the increasing solute gradient starts at a solute concentration substantially similar to a solute concentration of the first exchange media.

18. The method of claim 8, further comprising flushing the cell mass with the first exchange media until a first effluent has a composition substantially similar to the first exchange media.

19. The method of claim 8, further comprising flushing the cell mass with the second exchange media until a second effluent has a composition substantially similar to the second exchange media.

20. The method of claim 8, further comprising flushing the cell mass for a flushing time period.
